(19) United States
(12) Patent Application Publication
(10) Pub. No.: US 2002/0034534 A1
(43) Pub. Date: Mar. 21, 2002

(54) System and method for delivering a therapeutic agent over an extended period of time in conjuction with a receptor loading dose of the therapeutic agent

(76) Inventors: Deborah P. Barr, Richmond, VA (US); William H. Barr, Richmond, VA (US); Peter C. Coleman, Richmond, VA (US); John B. Vellines, Richmond, VA (US)

Correspondence Address: PENNIE AND EDMONDS, 1155 AVENUE OF THE AMERICAS, NEW YORK, NY, 100362711

(21) Appl. No.: 09/739,467

(22) Filed: Dec. 18, 2000

Related U.S. Application Data
(60) Provisional application No. 60/171,226, filed on Dec. 16, 1999.

(51) Int. Cl. A61K 9/00; A61K 9/14

(52) U.S. Cl. 424/424; 424/487

(57) ABSTRACT

The present invention provides systems and methods for administering a therapeutic agent to a patient in need an initial loading dose of the therapeutic agent administered in conjunction with a long-term maintenance dose of the therapeutic agent. The present invention discloses subcutaneous-implantable dosage forms capable of administering the long-term maintenance dose of the therapeutic agent in a steady state or zero order manner. Systems and methods for administering an initial loading dose of a therapeutic agent administered in conjunction with an extended release dosage form of the therapeutic agent, and optionally in conjunction with a receptor loading dose of the therapeutic agent, are useful in the treatment of various diseases and disorders.

This application claims priority under 35 U.S.C. §119 from U.S. Provisional Patent Application No. 60/171,226, filed Dec. 16, 1999.

### TECHNICAL FIELD

The present invention relates to a system and method for administering a therapeutic agent to a patient in need of such agent over an extended period of time in conjunction with an initial loading dose of a therapeutic agent. Specifically, the system and method of the present invention utilize at least one non-erodible, subcutaneous-implantable dosage form for administering the therapeutic agent over an extended period of time while the initial loading dose may be administered by any method known in the art of drug delivery. Advantageously, the non-erodible, subcutaneous-implantable dosage forms of the present invention provide a steady state or zero order release of the therapeutic agent over the lifetime of the subcutaneous-implantable dosage form thereby decreasing the number of times that the patient needs to be seen by a healthcare provider and increasing patient compliance for long-term therapeutic regimens. Diseases/disorders which may be beneficially treated by the system and method of the present invention include, but are not limited to the following: addictive disorders; psychiatric disorders; infectious diseases; cardiovascular diseases; respiratory diseases; inflammatory conditions; immune-based diseases; auto-immune diseases; bone diseases; and cancers.

### BACKGROUND OF THE INVENTION

Patient compliance is one of the most important aspects to disease/disorder treatment facing healthcare providers in their efforts to treat diseases/disorders that require an initial loading dose of a therapeutic agent followed by a long-term therapeutic regimen. In the past, healthcare providers have had to rely upon patients taking their medicine without supervision or in extreme cases healthcare providers have had to visit patients in their homes to supervise that the patients have taken their medicine. Long-term management of therapeutic regimens is therefore a burden on both the patient and the healthcare provider, which requires frequent trips/visits for the patient, frequent administration of the therapeutic agent and frequent refills of the therapeutic agent, resulting in high cost to the patient and healthcare provider. Thus, patient compliance in long-term therapeutic regimens is the weak link in treating diseases/disorders that can be eradicated or controlled by long-term therapeutic administration.

Additionally, several diseases/disorders that may be effectively treated through the use of long-term administration of a therapeutic agent will benefit from an initial loading dose of the therapeutic agent administered concurrently with the long-term therapy. In particular, addictive disorders will benefit from such a combination. The initial loading dose can serve to help the patient overcome the initial withdrawal symptoms, which can be quite severe, and the long-term therapy will held to keep the recovering patient from reverting to drug use.

In particular, the long-term treatment of patients addicted to narcotics is a pressing healthcare concern. Addiction to heroin and other narcotics is a serious healthcare problem in the United States. Federal figures in 1998 estimate that there are more than 800,000 opioid addicts in the United States, and this number appears to be growing (McCaffery, B., Publications of the Department of Justice, U.S. Navy, 1993, 3). Current methods of treating narcotic addiction can be divided into two groups: (1) opiate agonist treatment (replacing the narcotic that an addict has been using with a medically sanctioned, safer narcotic) and (2) “drug-free,” non-agonist treatment. Narcotic antagonists (also known as narcotic receptor blockers), discussed later, have been used to improve the outcome of the second type of treatment.

Narcotic or opiate agonists used in the treatment of narcotic addiction include methadone, LAAM (L-Alpha-Acetyl-Methadol), and most recently buprenorphine. Methadone was first described by Dole and Nyswander in 1965 as a method of helping heroin addicts to decrease their use, thereby decreasing crime and other costs to society associated with heroin use. Today in the United States, it is estimated that there are 112,000 patients on methadone maintenance. LAAM, and more recently, buprenorphine, have been studied as narcotic substitutes for treating narcotics addiction. These substitutes have been effective at meeting their stated goals, including decreasing heroin use and decreasing crime and the spread of HIV and other infections associated with drug use. Many physicians, patients, and members of the general public, however, find the approach of substituting other addictive narcotics for heroin to be a less than ideal form of long-term treatment.

Drug-free treatment programs are found throughout the United States and represent the most common form of treatment for addiction. After detoxification, the patient is provided with counseling in the form of either individual or group therapy. The patient is usually expected to make a commitment to avoid alcohol and other mood altering drugs, and to attend Narcotics Anonymous meetings or other 12-step support groups. Sometimes the patient is housed in a supervised recovery residence or therapeutic community to enhance recovery rates. Even with this intensity of treatment, however, long-term recovery success rates are low and relapse is altogether common.

Narcotic antagonists (naltrexone, naloxone, cyclazocine, and nalmefene) have been used since 1978 to try to enhance the success rates of drug-free treatment programs. These compounds work by blocking the opiate receptors for narcotics in the brain, thereby preventing the patient from feeling any of the effects of the narcotic if the patient relapses back to narcotic use. When faithfully administered, narcotic antagonists are extremely effective in preventing relapse and treating narcotics addiction.

The most commonly used drug in this category is naltrexone, which is approved by the FDA in the oral dosage form for use as a narcotic antagonist. When the recommended dose of 50 mg is taken orally, it provides complete blockage for 24 hours. It can be administered three times per week to ensure the patient's compliance with drug-free treatment. Studies have confirmed naltrexone's effectiveness, especially when patients are subject to court ordered treatment, or in other similar situations in which the addict is highly motivated (Tai, et al. NIDA Treatment Workshop “Nalterxone—An Antagonist Therapy for Heroin Addiction”, Nov. 12-13, 1997). However, in real world settings, naltrexone's effectiveness is very low. Involuntary or purposeful non-compliance with self-administration by patients has limited the utility of orally administered naltrexone. Accordingly, the drug has been used infrequently in most treatment settings.

To increase the effectiveness of narcotic antagonists, a number of delivery systems have been tried:

1. Naltrexone pellets—Gooberman and Malmberg have developed an implanted pellet that contains 1 gm of naltrexone (“Depot Naltrexone Implants for Opiate Dependence: Clinical Experience, Plasma Levels, Opiate Challenge Results in over 200 Patients”, International Council on Alcoholism and Addiction Annual Meeting, Cairo, May 17-22, 1997). It provides the naltrexone in a tightly compressed pellet, which is inserted under the skin and slowly releases the drug over a 40-80 day period. The release rate has not been well established but it appears to be inefficient and to vary widely between patients. It is not an FDA-approved product. One of the problems with this delivery system is that the pellets tend to liquefy towards the end of their lifespan, making removal all but impossible. Moreover, being a first order release system, it is unlikely that the pellet will be able to release naltrexone over an extended period of time without creating a significant risk of toxicity because of the high initial levels that would be required. With a first order release system, the only way to increase the duration of the effect is to increase the total dose and accordingly, the size of the pellet. A dose higher than 1000 mg poses a number of problems. First, there is a greater risk of toxicity if the pellet disintegrates. Second, a bigger pellet presents a significant challenge to subcutaneous insertion and acceptance by patients.

2. Naltrexone microspheres—In this system, microspheres containing naltrexone are injected into patients intramuscularly. They were originally developed through the National Institute of Drug Abuse (NIDA) around 1970 and were studied in some patients. The microspheres were found to cause problems with inflammation (Chiang, et al., Clinical Pharmacology and Therapeutics, 1984, 36, 704). A major problem with the microspheres is that the naltrexone cannot be removed from the body once it has been injected. Thus, should a patient require narcotics for medical reasons such as in the case of a serious accident or broken bone, he or she would be unable to derive any benefit from narcotic-type painkillers as long as the microspheres were still active in the body. Also, the inability to remove naltrexone microspheres is a problem if there is an allergic response to naltrexone, which is known to occur.

Microspheres have been used in small trials to treat alcoholics. Injection into the gluteus maximus produced blood levels above 1 ng/ml for about 20 days. The overall release was not zero-order. Rather, it declined steadily over about 40 days (Kranzler, et al., Alcoholism: Clinical and Experimental Research, 1998, 22, 1074).

Drug Abuse Sciences recently announced the beginning of clinical trials involving a microsphere injectable dosage form (NALTREL®) that would deliver naltrexone over a 30-day period (http://www.drugabusesciences.com/products.asp). The problems inherent in this system have been mentioned above. Additionally, naltrexone is depleted at a rate that varies among patients. This variability among patients could create a significant problem with drug buildup and risk of toxicity because subsequent injections need to be given to ensure complete blockage in all patients, especially those whose blood levels of naltrexone decrease the fastest. Patients who have slower metabolism, however, will still have large amounts of naltrexone in their bloodstream, and subsequent injections may possibly push them into an overdose situation.

3. Biodegradable implant—A long-acting implant has been designed and patented by Sidman (U.S. Pat. No. 4,351,337). This implant incorporates the drug into a poly-alpha-amino acid structure. The structure is biodegradable and slowly dissolves in the body. The drug may be incorporated into a matrix of the poly-alpha-amino acid and then slowly released by hydrolysis, or it may be contained within a closed structure of the poly-alpha-amino acid and then diffuse out. Alternatively, the drug may be released through a combination of both hydrolysis and diffusion. The patent specifically mentions that narcotic antagonists, including naltrexone, may be used with such a delivery system. Examples 3,4 and 5 disclose that naltrexone could be used with such a system but they do not disclose or suggest that the release can be done in a zero order.

In U.S. Pat. No. 4,450,150, Sidman employs an implantable poly (glutamic acid-ethyl glutamate) structure. Specifically, in example 1 he describes a rod-shaped implant containing 21.1 mg of naltrexone. He was able to demonstrate a near zero order release rate for 24 days and extrapolated that the drug release may last longer since only one third of the naltrexone had been released within the twenty-four days. There are significant shortcomings with this invention, however. First, the implant has only been shown to be effective for 24 days. Second, the implant is by nature an erodible device. When it incorporates the drug into the poly (glutamic acid-ethyl glutamate) structure, the release cannot be zero order because the surface area of the implant gets smaller and smaller over time. While this structure has demonstrated a 24 day release, the structure is not suitable for long-term, zero order release. Alternatively, the drug may be enclosed within the poly (glutamic acid-ethyl glutamate) structure. At some point, the structure must erode and then the release rate cannot be zero order. Third, the fact that the implant is designed not to be removed means that subsequent implants can create a toxicity problem. At the end of the delivery cycle there will always be a fall off in the release rate. At some point, the release rate falls below the therapeutic value or the minimum effective concentration (MEC). At this point, if patients require more naltrexone, it could be dangerous to insert another implant because the dosage released by the new implant and the residual dosage from the old one have an additive effect that could push concentrations in the bloodstream to a toxic level.

4. Transdermal delivery system—A transdermal delivery system has been developed by Glier, et al. (U.S. Pat. No. 4,806,341) which promises to deliver doses of narcotic antagonists such as naloxone (Narcan) through the skin. This system, however, would not address any of the effectiveness criteria identified above since patients could easily remove it. It therefore would not represent a significant improvement over the oral administration of naltrexone in terms of effectiveness and patient compliance.

Thus, based upon the prior disclosure, there exists a need for a system and method for delivering a therapeutic agent over a extended period of time in conjunction with an initial loading dose of the therapeutic agent. While the background art has been examined particularly in the area of narcotic antagonists, this examination is in no means considered to be limiting but rather serves to illustrate the need for the system and method of the present invention.

### SUMMARY OF THE INVENTION

The system and method of the present invention address the above-described shortcomings in prior art delivery systems for delivering therapeutic agents requiring an initial loading dose administered in conjunction with a long-term maintenance delivery of the therapeutic agent. In particular, the system and method of the present invention are useful for the administration of naltrexone and other narcotic antagonists in the treatment of opioid addiction, alcoholism and other addictions.

One object of this invention is to provide a system whereby a single administration of a therapeutic agent will provide sufficient plasma levels of the agent in patients to treat the disease/disorder for at least six months. Thus, the drug delivery system of this invention must be sufficiently efficient to provide effective levels of a therapeutic agent for at least six months with a dose of the drug that can be practically incorporated into a non-erodible, subcutaneous-implantable dosage form. This duration of release is critical because the usual poor compliance of patients, particularly for products used to treat addiction, often results in relapse within the first 6 to 12 months. A preferred system of the present invention exists whenever the implantable dosage form is in the form of a hydrogel tube. The preferred hydrogel tube can be modified so that the system releases the therapeutic agent more quickly or slowly depending upon the disease/disorder to be treated.

A further, object of the present invention utilizes the non-erodible, subcutaneous-implantable dosage form of a therapeutic agent of the present invention in conjunction with a loading dose of the therapeutic agent. The loading dose may take the form of any dosing method known to one of skill in the art such as, for example, oral, parenteral, intravenous and subcutaneous implant.

Another objective of this invention is to provide the optimal dose and rates of delivery of a therapeutic agent such as, for example, naltrexone and other narcotic antagonists that will produce the minimum effective concentration (MEC) of the antagonist in a particular patient's bloodstream for the required duration of time. A system according to this invention must define at least two treatment phases, each of which has its own minimum serum concentration value and therefore its own delivery rates for any implantable device. The defined optimal release rate profile for a particular agent such as, for example, the narcotic antagonist naltrexone, can be derived through a method that combines different types of dosage forms including oral, subcutaneous and intramuscular injections, and subcutaneous inserts of non-erodible delivery devices such as hydrogel implants, silastic tubes or osmotic pump implants that are capable of providing multiple release rates.

### BRIEF DESCRIPTION OF THE DRAWINGS

FIG. 1—illustrates the in vitro release rates of naltrexone from various hydrogel, subcutaneous-implantable dosage forms having different percent values of Equilibrium Water Content (% EWC).

### DETAILED DESCRIPTION OF THE INVENTION

The present invention describes a system and method for administering a loading dose of a therapeutic agent concurrently with a subcutaneous-implantable dosage form, which releases the therapeutic agent over an extended period of time, to a patient in need of such a therapeutic regimen. Diseases/disorders which may be beneficially treated by the system and method of the present invention include the following: addictive disorders such as, for example, narcotic addiction, alcohol addiction and craving and nicotine addition; psychiatric disorders; infectious diseases such as, for example, post-operative infections; cardiovascular diseases; respiratory diseases such as, for example, tuberculosis; inflammatory conditions such as, for example, rheumatoid arthritis; immune-based diseases such as, for example, hypersensitivity; auto-immune diseases such as, for example, multiple sclerosis; bone diseases; and tumor causing and non-tumor causing cancers.

The subcutaneous-implantable dosage forms of the present invention can take the form of any such dosage form known in the art such as, for example, non-erodible implants. Subcutaneous-implantable dosage forms provide many advantages for long-term drug administration for the following reasons:

1. They administer the drug to the patient in a defined manner precluding the need for active participation by the patient in the administration of the drug;

2. They administer the drug in a dose and dose rate to produce drug levels higher than the minimum effective concentration (MEC) throughout the duration of treatment;

3. They administer the drug in a dose and dose rate to produce drug levels close to the MEC to minimize any chances of toxicity and to maximize the efficiency and cost of the system;

4. They minimize the first pass exposure to the liver, decreasing the potential for hepatoxicity; and

5. They decrease intestinal and hepatic first pass metabolism increasing systemic availability.

Specifically, non-erodible subcutaneous-implantable dosage forms provide many advantages over erodible forms for the following reasons:

1. They can be removed by trained medical personnel in the event of an emergency in which other therapeutic agents must be administered, which are not compatible with the drug being administered by the subcutaneous dosage form;

2. They can be removed when the delivery rate falls below the MEC so that subsequent implants can be inserted without any risk of unnecessary drug buildup and toxicity; and

3. They can be removed if an allergic reaction to the drug occurs.

Additionally, the subcutaneous-implantable dosage forms may take the form of hydrogels, silastic tubes and osmotic devices. Preferred dosage forms of the present invention are non-erodible hydrogels. Small cylindrical implants with thin walls that can predictably control the rate of drug release can be constructed using the class of compounds referred to as hydrogels. These compounds are biocompatible; they resist biodegradation and do not support microbial growth. They consist of various esters of methacrylic acid such as, for example, hydroxyethyl methacrylate and hydroxypropyl methacrylate that are copolymerized to form a polymer matrix that incorporates water. The water-filled pores of the matrix allow drug molecules to diffuse from the interior to the surrounding bulk fluid. When the bulk fluid is the interstitial fluid surrounding the cells in the body, the drug released is expected to be absorbed into the blood circulation, thence to be delivered to the sites of action in the body where the drug will exert its pharmacologic effects.

It is possible to adjust the Equilibrium Water Content (EWC) to obtain specified release rates. The following illustration of the invention was developed using the procedures described by Kuzma (Kuzma, et al., “Long-term drug delivery from subcutaneous hydrogel implants”, Proceedings, Controlled Release Society Conference on Protein/Peptide Controlled-Release Delivery. Baltimore Md. Aug. 21-22, 1996 and Kuzma, et al., U.S. Pat. No. 5,266,325). Homogeneous Polymers of 2-hydroxymethyl acrylate (HEMA), methacrylic acid, and 1,1,1, Trimethylol propane trimethyl acrylate (TMPTMA) were prepared, as described by Kuzma, such that the relative concentrations are in the range needed to produce certain values of the equilibrium water content (EWC). These EWC values result in in vitro rates in the range expected to produce the desired concentration-time profile.

The initial loading dose of therapeutic agent in the system and method of the present invention may take the form of any method of drug administration known to one of skill in the art such as, for example, oral, parenteral, intravenous or subcutaneous implant. The preferred method of administration will vary upon the disease/disorder to be treated and the therapeutic agent chosen for the treatment regimen.

The method of the present invention determines the desired dose of therapeutic agent needed to achieve the desired plasma levels of the therapeutic agent as well as the corresponding rates of delivery required to effectively treat the disease/disorder for the six to twelve month life of the subcutaneous-implantable dosage form. There are at least two phases of drug administration in the method of the present invention: the Receptor Loading Dose (RLD); and Maintenance Phase (MP). Optionally, the method of the present invention may include a third phase known as the Initial Loading Dose (ILD) at the initiation of dosing. Further discussion outlining the process to determine the optimal drug delivery rates for each of these phases will be presented for the narcotic antagonist, naltrexone.

Phase I (ILD)—Days one to two. Immediately after implants are administered, there may be a lag time of 1 or 2 days for the drug to diffuse through the rate-controlling membrane and surrounding tissues before it reaches the bloodstream. During this time period, a rapidly available source of the drug can be administered as an initial loading dose to quickly produce effective levels until the drug from the implants reaches effective levels. This initial loading dose can be given orally for one or two days. Alternatively, one of the implants could be coated with a rapidly dissolving drug coat.

Phase II (RLD)—Days two to thirty. During this period, the dosage of drug is lower than that in Phase I but higher than that in Phase III. The higher serum levels of this phase serve two purposes. First, for the particular therapeutic agent chosen, the long-term maintenance dose releases drug slowly at a constant (zero order) rate. This produces the most efficient, longest acting means of delivering the drug. It has the disadvantage, however, that the plasma level will slowly rise until it reaches a plateau, which is referred to as steady state. The time to reach steady state is a function of the drug's half-life, a measure of the time it takes for half of the drug to be eliminated from the body. It takes approximately six half-lives to reach steady state for any drug. As will be shown below the narcotic antagonist, naltrexone has an effective half-life of almost 100 hours. So it will take about 20 days before steady state is reached for naltrexone. During this time it is essential that an additional source of drug be administered as a receptor loading dose to continue to saturate the drug receptors. A second reason for this loading dose, particularly for drugs used to treat addictions, is to prevent cravings and other withdrawal symptoms.

Phase III (MP)—Months one to Twelve. During this period, the MEC is lower and the system of the present invention can be tailored to maintain plasma concentration above the MEC at steady state in an efficient zero order manner.

The method for optimizing drug delivery during each of these phases are based upon an analysis of the pharmacokinetic and pharmacodynamic properties of the therapeutic agent administered and clinical observations. The following discussion includes first, a description of the method of delivering the optimal drug delivery rate to allow a practical dose of drug, administered one time, to maintain adequate plasma levels from six to twelve months. This is followed by a description of how the drug delivery system can be further optimized, based upon a pharmacokinetic model of the interaction of the drug with its receptor binding sites in the body and clinical observations.

After adequate blood levels have been achieved during Phase I and Phase II, the ideal delivery system should provide a prolonged constant blood level above the MEC. An objective of the invention is to provide a drug delivery system that will deliver the drug at an optimal rate and optimal levels to ensure that the therapeutic agent will have the desired therapeutic effects for the maximum duration of time. It is generally agreed that the ideal delivery system would provide a zero order input, that is, a constant rate, in the MP. This MP (Phase III) should provide a zero order input sufficient to maintain steady state plasma levels above the MEC of the selected therapeutic agent. This parsimonious, “drug-sparing” input will allow a single administration of the dosage form to provide effective maintenance levels for the longest period possible. Ideally, it would be advantageous to have a system that would release drug for a year or more. At least a six month period is possible based upon an interpretation of the pharmacokinetic data available in the literature for naltrexone, a preferred embodiment of this invention.

The following discussion illustrates the steps necessary to determine an MEC for a therapeutic agent, according to the present invention. Additionally, the following discussion illustrates how one of skill in the art may use the calculated MEC data to formulate a dosage form that will release the therapeutic agent over an extended period of time to a patient in need of such therapeutic agent. The following discussion, while specifically related to narcotic antagonists and more specifically the narcotic antagonist, naltrexone, is not intended to limit and shall not be construed to limit the system and method of the present invention but rather serves to illustrate the novelty and utility of the system and method of the present invention. It is expected that one of ordinary skill in the art would be able to apply the system and method described herein to a wide variety of therapeutic agents.

### DETERMINATION OF THE DOSAGE RATE FOR PHASE III (MP)

A reinterpretation of the pharmacokinetic data available in the literature, which is disclosed below, utilizing the concepts of the present invention, demonstrate that a therapeutic agent can be delivered in a substantially zero order manner for a period between about six months and about twelve months with the system and method of the present invention.

The zero order rate of delivery (D/T) of the dose (D), delivered constantly over a given period of time (T), that is required to reach a desired steady state, minimum effective plasma concentration (MEC), for a given plasma clearance (CL), is given by the well-known equation:

(D/T)=CL×MEC

The prior art states that the plasma half life of naltrexone, a well-known narcotic antagonist, is about 2 to about 10 hours and the corresponding plasma clearance is about 2 to about 4 L/min (Chiang, et al., Clinical Pharmacology and Therapeutics, 1984, 36, 704 and Willette, et al., eds., “Narcotic Antagonists: Naltrexone Pharmacochemistry and Sustained-Release Preparation”, NIDA Research Monograph 28, 1981). Therefore, the maintenance dosage rate necessary to achieve a MEC of 2 ng/ml may be calculated using the equation:

D/T=(3 L/min×60 min/hr×24 hr/day)(2 mcg/L)=8.64 mg/day

Such a dosage rate would require a dose of 1.56 g in a zero order controlled release implantable dosage form to provide effective levels for six months. This required dose is greater than possible with current implantable dosage form technologies. A reinterpretation of several prior art studies utilizing the teaching of the present invention demonstrates that the dosage rate at steady state is considerably less, thereby allowing a given dose to provide effective levels for a greater period of time.

Important new information was provided by Lee (Lee, et al., J. Nucl. Med. 1998, 29, 1207). Using new positron emission tomography (PET) technology with ¹¹C-carbfentenil, they recently disclosed that when the μ-opiate receptors in the brain are occupied by naltrexone (thus blocking the effects of opioids), the dissociation of naltrexone from the receptor followed a monoexponential decline corresponding to a half-life of approximately 72 hours, which corresponds to a first-order rate constant of 0.0096/hr. The time course of occupancy of naltrexone on the opioid receptors in the human brain was found to be much longer than would have been predicted by most previous blood level studies. The percent blockade of opiate receptors by 50 mg of oral naltrexone was 90 to 100% for 48 hours, followed by the monoexponential decline. Lee points out that this is similar to the long half-life of 96 hours observed by Verebey and coworkers (Verebey, et al., Clinical Pharmacology and Therapeutics 1976, 315) in the third exponential phase of the naltrexone plasma-concentration time curve (at 24 to 72 hours following a 100 mg dose of naltrexone).

It is noteworthy that Verebey, et al., did not associate the long terminal elimination phase with the opioid receptors. They disregarded the terminal half-life in their assessment of the dosage rate (D/T) required to produce an effective plasma level in the maintenance phase. In a subsequent paper, they estimated D/T to be 11.8 g/kg/hr (Kogan, et al., Res. Com. Chem. Path. and Pharmacol., 1977, 18, 29). This corresponds to a daily dose of 20.4 mg/day for a 72 kg person, or a total dose of 3.67 grams for six months. Again this is not possible with currently available drug delivery systems.

A reinterpretation of these data, utilizing the disclosure of the present invention is based on the following assumptions:

1. The terminal plasma concentration observed by Verebey, et al., represents distribution equilibrium between the brain opioid receptors and the systemic plasma compartment, which occurs in the terminal exponential phase after a single dose, and at steady state after continuous (zero order) dosing;

2. Therefore, the terminal half-life (t_{½}(3)) in the plasma represent the half-life of naltrexone dissociating from the receptors. This concept provides an alternative way, using only plasma concentration-time data, to obtain the receptor dissociation half-life for other narcotic antagonists that is much simpler and less costly than the ¹¹C PET method used by Lee, et al. In fact it can be used for any drug in which the dissociation from the receptor is the slowest distribution step;

3. As the brain receptors and systemic plasma are in distribution equilibrium at the time that the terminal exponential phase occurs, the volume of distribution of the receptor compartment is the same as the volume of distribution for the terminal phase (V₃);

4. The clearance term representing loss of naltrexone from the brain receptors is determined using the following equation,

Cl₃=V₃×k₃

where V₃ and k₃ are calculated from the plasma concentration time curve; and

5. When steady state distribution equilibrium occurs, the rate of dosing to the receptors will be equal to the rate of loss of antagonist from the receptors. The Maintenance Dosage Rate (D/T) is calculated by the following equation,

D/T=MEC×V₃×k₃

The teachings of the present invention show that the estimates of plasma clearance and therefore the maintenance dose needed to reach a given MEC are actually much lower than the rates taught by the prior art. Further, the teachings of the present invention demonstrate that the time needed to reach steady state equilibrium using a zero order maintenance does is longer than the times taught in the prior art.

The present invention teaches that to achieve at least 98% blocking of the opioid receptors, with the inherent variability between patients, a Maintenance Dose Rate of from about 250 to about 1500 microgram/day is necessary.

### Phase II Concentration Requirements

Additionally, the teachings of the present invention take into account the fact that steady state does not occur immediately but requires a period of time to reach distribution equilibrium. A period of approximately 3.3 half-lives is necessary to achieve a 90% receptor occupancy level. For naltrexone, if the maintenance rate is set to reach the plasma level corresponding to a 90% occupancy level at distribution equilibrium, the time to reach this level is calculated as 3.3×72 hr, which is about equal to about 10 days. To reach a 98% occupancy with naltrexone will require about 20 days. This period of time necessary to reach distribution equilibrium is the reason that the Receptor Loading Dose of the present invention is necessary to provide a new and useful system and method for delivering therapeutic agents.

### Variable Rate Requirements

To provide sufficient blocking levels over the entire period required for the maintenance dose to reach steady state, two or three types of inserts could inserted simultaneously. One would produce a higher initial rate for a short period of time (e.g., 2 weeks to 2 months). This would serve as the receptor loading dose to ensure that plasma and brain levels would be significantly above the 100% receptor occupancy level during the time (10 to 20 days) that it takes for the implants with the slower maintenance rate to reach steady state distribution levels in the plasma and the brain.

There are additional advantages in maintaining higher levels during the first 1 to 2 months. During this period, the addict is most likely to test the blocking power of the implant, perhaps using larger than usual narcotic doses. It is critical during this conditioning period that the patient is prevented from overcoming the blockade of the receptor. Moreover, if the opioid receptors are not completely blocked during the initial treatment phase (Phase I and II), some patients receiving naltrexone may use heroin to reduce craving. The following clinical study in one such subject illustrates this problem.

### EXAMPLE 1

K.R.—is 41 year old white male who has had a problem with heroin addiction for about six years. A URD was performed (ultra rapid detox or anesthesia assisted opiate detoxification) and a pellet of naltrexone was inserted simultaneously which contains 1 g of naltrexone and 12.5 mg of triampcinalone. In other patients, this pellet has been found to completely block the euphoric effects of self administration of heroin or any other narcotics, even in large doses. However, the patient had some persistent withdrawal symptoms following his detoxification and used some heroin. He reported that this made him feel more comfortable with his withdrawal symptoms although he did not experience a euphoria. He continued to do this on a regular basis and said that the heroin would reliably decrease his withdrawal symptoms, particularly low energy, decreased appetite, and insomnia. An investigation was conducted to compare the effectiveness of the naltrexone pellet with the higher blood levels of naltrexone that can be achieved with oral administration.

With only the naltrexone pellet in place providing a low naltrexone blood level, an intravenous (IV) administration was conducted over a 30 minute period comprising a series of six injections, three of which were normal saline and three of which were morphine sulfate for a total dose of 50 mg. Clinical evaluation indicated mild relief of withdrawal symptoms but no euphoria. Administration of a 1 mg of naloxone (Narcan) was given to reverse the effects of the total of 50 mg morphine resulting in a return to a baseline level of withdrawal symptoms. Then in Phase II of the experiment, the patient received 100 mg of oral naltrexone. Approximately five hours later, when the Naltrexone blood level was higher, the same experiment was repeated. In this second phase, four injections, two with Morphine sulfate (125 mg) and two with normal saline were administered. This time there was no subjective or objective change in any withdrawal symptoms and no euphoria. When we reversed the Morphine with naloxone (Narcan 2 mg) there was no change in his withdrawal symptoms. With the second experiment, a higher dose of naltrexone was delivered by the oral medicine as a supplement to the pellet. In this case, there was no override of the blockage effects and no inducement of withdrawal symptom relief or euphoria, even with a dose of 50 mg of Morphine sulfate.

In this study, a higher amount of naltrexone was necessary to overcome the craving even though a euphoric high was blocked by a lower dose of naltrexone. There are several possible mechanisms that might explain these results. One is that craving may be related in part to other sites of action that require higher levels of naltrexone to block them. An alternative explanation is that the craving still occurs at a higher level of occupancy of the opiate receptors than the “high” effect. The data of Verebey, et al., suggest that the opiate high rating was blocked longer after a single dose than the “liking” rating which in turn correlated with the amount the subjects were willing to spend for a heroin dose (Verebey, et al., Clinical Pharmacology and Therapeutics 1976, 315). These results suggest that higher levels of naltrexone are needed for the latter effects, regardless of the mechanism. Since the extinguishment of conditioned responses is so critical during the early phase of treatment, a multi-phase drug delivery system is necessary (O'Brien, et al., “Naltrexone in a Behavioral Treatment Program”, NIDA Research Monograph 1976, 136; O'Brien, et al., J. Clin. Psychiatry, 1984, 45, 53; and Tai, et al., NIDA Treatment Workshop “Nalterxone—An Antagonist Therapy for Heroin Addiction”, Nov. 12-13, 1997). Phase II (months 1 to 2) provides higher levels than the minimum needed to prevent narcotic highs. This ensures that attempts to use high doses to override the naltrexone blockage will be thwarted. The higher levels may be necessary in some patients to overcome craving symptoms that occur early during treatment while the extinguishment of conditioned response is being initiated in the behavioral component of the treatment program. For pharmacokinetic reasons, the higher levels are also needed until the steady state distribution levels are reached through the slower maintenance dose. A subcutaneous product that delivers drug at a zero order rate of 0.56 mg/day will produce a steady state level of about 7.5 ng/ml, which is greater than the concentration required, when equilibrated with brain levels, to produce complete receptor occupancy after about 10 to 30 days.

### Phase I Concentration Requirements

To reach effective blood levels immediately, a “loading dose” of 50 to 100 mg may be given orally for one to three days or it may be incorporated into the subcutaneously administered dosage form. The amount of loading dose would be given by the formula:

LOADING DOSE=VOLUME OF DISTRIBUTION×MEC

This amount could be coated on the outside of the cylindrical insert for immediate release to provide drug for the first few days.

The above calculations for naltrexone are based upon values from the literature on clearance values and the MEC, as well studies which relate brain occupancy to observed clinical responses. The combination of extensive accumulated data and our conceptual approaches to provide optimal input rates for naltrexone represents an advance in the state of the art for providing long lasting therapeutic effects. This general method of optimizing delivery to maximize the time during which a given dose provides an effective amount of drug is applicable to any narcotic antagonist, including but not limited to naltrexone, nalmefene, and cyclozine. The antagonist is administered subcutaneously in an erodible or non-erodible implantable insert in which the rate of delivery can be controlled and predicted by in vitro release rates, such as hydrogels, silastic tubes or osmotic devices. A preferred embodiment of this new method of optimizing naltrexone administration follows.

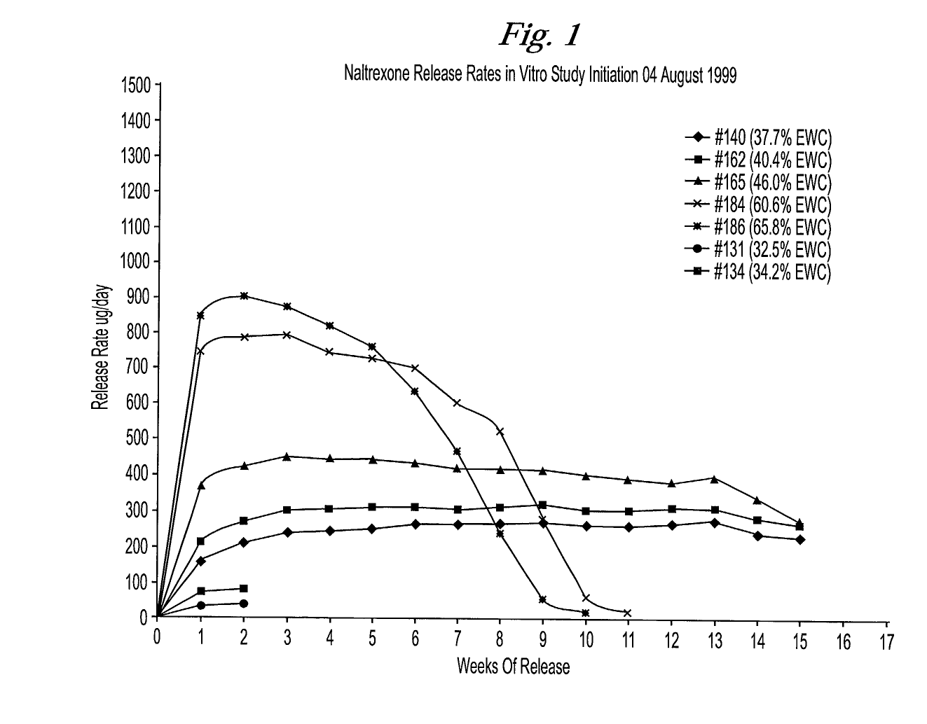

Cylindrical polymer tubes, as above described, were produced from the hydrogel compounds and filled with ±5 mg of anhydrous naltrexone base (Mallinkrodt) and stearic acid, NF. The tube was sealed at both ends to form an implant approximately 35 mm in length. The in vitro release of hydrogels of varying EWC (37.7%; 40.4%; 46.0%; 60.6%; 65.8%) was measured by placing the Hydrogels in elution vials containing approximately 10 ml of elution medium at 37° C. and analyzed by a validated HPLC assay. Three implants of each composition were tested. The mean release rates were obtained weekly. The mean rates plotted as a function of time are shown in FIG. 1. The final delivery system involves the subcutaneous implantation of one or more implants for the loading dose (Phase II) and implantation of one or more implants for long term maintenance (Phase III). One of these implants may be coated with an immediate initiating dose (Phase I). Alternatively, the initiating dose can be given orally, e.g., 50 to 100 mg naltrexone tablets for days 1-2.

An example of ranges of values that would constitute this invention is shown in the table below:

| PERIOD | PERIOD 1 | PERIOD 2 | PERIOD 3 |
|---|---|---|---|
| FUNCTION | initiating dose | receptor | maintenance |
| | | loading/ | dose |
| | | relapse | |
| | | prevention | |
| DURATION | 1-2 DAYS | 2-60 DAYS | 180 to |
| | | | 360 DAYS |
| TARGET BLOOD | 20-60 ng/ml | 4-10 ng/ml | 1-4 ng/ml |
| LEVELS | | | |
| DOSE | 50-100 mg | 65 mg | 65 mg |
| | Oral | (1 tube) | (1 tube) to |
| | 5-10 mg | | 230 mg |
| | parenteral | | (4 tubes) |
| RATE | Immediate | 500-2000 | 250-1500 |
| | (bolus) | μg/day | μg/day |

The final composition of the implants is determined by experimentally measuring the plasma concentrations that result from the tested implants in volunteers. Morphine challenges can be done in selected volunteers/patients to validate clinical effectiveness and determine the rang of naltrexone concentration values that serve as the MEC.

These data are used to optimize the final dosage form. This system allows the overall dosage regimen to be individualized by varying the number and rate of the individual dosage forms. In this way, treatment can be tailored to the needs of the individual patient.

From the foregoing description, one of ordinary skill in the art can easily ascertain the essential characteristics of this invention and, without departing from its spirit or scope, can make various changes and modifications in the invention to adapt it to various usages and conditions, such changes and modifications are hereby covered by the present invention.

1. A system for administering a therapeutic agent to a patient in need of the agent, comprising:
at least one receptor loading dosage form containing the therapeutic agent; and
at least one extended-release dosage form containing the therapeutic agent, wherein the receptor loading dosage form and the extended-release dosage form are administered substantially concurrently.

2. The system according to claim 1, wherein the at least one receptor loading dosage form is selected from the group consisting of oral dosage forms, parenteral dosage forms (intramuscular or subcutaneous), intravenous dosage forms, a coating on the at least one extended-release dosage form and implantable dosage forms.

3. The system according to claim 1, wherein the at least one extended-release form is a non-erodible, implanted, subcutaneous dosage forms.

4. The system according to claim 3, wherein the non-erodible, implanted, subcutaneous dosage form is selected from the group consisting of hydrogels, silastic tubes and osmotic pumps.

5. The system according to claim 4, wherein the at least one extended release dosage form is a non-erodible, implanted, subcutaneous, hydrogel dosage form.

6. The system according to claim 5, wherein the at least one extended release dosage form is a non-erodible, implanted, subcutaneous, hydrogel dosage form fashioned from a co-polymer of various esters of methacrylic acid.

7. The system according to claim 1, wherein the therapeutic agent is useful for treating diseases and disorders selected from the group consisting of addictive disorders, psychiatric disorders, infectious diseases, cardiovascular diseases, respiratory diseases, inflammatory conditions, immune-based diseases, auto-immune diseases, bone diseases and cancers.

8. The system according to claim 7, wherein the disorder is selected from the group consisting of narcotic addiction, alcohol addiction and craving and nicotine addiction.

9. The system according to claim 8, wherein the disorder is narcotic addiction.

10. The system according to claim 9, wherein the therapeutic agent is a narcotic antagonist.

11. The system according to claim 10, wherein the narcotic antagonist is selected from the group consisting of naltrexone, naloxone, cyclazocine and nalmefene.

12. The system according to claim 11, wherein the narcotic antagonist is naltrexone.

13. A system for administering a therapeutic agent to a patient in need of the agent, comprising:
at least one initial loading dosage form containing the therapeutic agent;
at least one receptor loading dosage form containing the therapeutic agent; and
at least one extended-release dosage form containing the therapeutic agent, wherein the at least one initial loading dosage form, the at least one receptor loading dosage form and the at least one extended-release dosage form are administered substantially concurrently.

14. The system according to claim 13, wherein the at least one initial loading dosage form and the at least one receptor loading dosage form are independently selected from the group consisting of oral dosage forms, parenteral dosage forms (intramuscular or subcutaneous), intravenous dosage forms, a coating on the at least one extended-release dosage form and implantable dosage forms.

15. The system according to claim 14, wherein the at least one extended-release form is a non-erodible, implanted, subcutaneous dosage forms.

16. The system according to claim 15, wherein the non-erodible, implanted, subcutaneous dosage form is selected from the group consisting of hydrogels, silastic tubes and osmotic pumps.

17. The system according to claim 16, wherein the at least one extended release dosage form is a non-erodible, implanted, subcutaneous, hydrogel dosage form.

18. The system according to claim 17, wherein the at least one extended release dosage form is a non-erodible, implanted, subcutaneous, hydrogel dosage form fashioned from a co-polymer of various esters of methacrylic acid.

19. The system according to claim 13, wherein the therapeutic agent is useful for treating diseases and disorders selected from the group consisting of addictive disorders, psychiatric disorders, infectious diseases, cardiovascular diseases, respiratory diseases, inflammatory conditions, immune-based diseases, auto-immune diseases, bone diseases and cancers.

20. The system according to claim 19, wherein the disorder is selected from the group consisting of narcotic addiction, alcohol addiction and craving and nicotine addition.

21. The system according to claim 20, wherein the disorder is narcotic addiction.

22. The system according to claim 21, wherein the therapeutic agent is a narcotic antagonist.

23. The system according to claim 22, wherein the narcotic antagonist is selected from the group consisting of naltrexone, naloxone, cyclazocine and nalmefene.

24. The system according to claim 23, wherein the narcotic antagonist is naltrexone.

25. A method of treating a patient in need of a therapeutic regimen, comprising the following steps:
administering at least one receptor loading dosage form containing the therapeutic agent; and
administering at least one extended-release dosage form containing the therapeutic agent, wherein the at least one receptor loading dosage form and the at least one extended-release dosage are administered substantially concurrently.

26. The method according to claim 25, wherein the at least one receptor loading dosage form is selected from the group consisting of oral dosage forms, parenteral dosage forms (intramuscular or subcutaneous), intravenous dosage forms, a coating on the at least one extended-release dosage form and implantable dosage forms.

27. The method according to claim 26, wherein the at least one extended-release form is a non-erodible, implanted, subcutaneous dosage forms.

28. The method according to claim 27, wherein the non-erodible, implanted, subcutaneous dosage form is selected from the group consisting of hydrogels, silastic tubes and osmotic pumps.

29. The method according to claim 28, wherein the at least one extended release dosage form is a non-erodible, implanted, subcutaneous, hydrogel dosage form.

30. The method according to claim 29, wherein the at least one extended release dosage form is a non-erodible, implanted, subcutaneous, hydrogel dosage form fashioned from a co-polymer of various esters of methacrylic acid.

31. The method according to claim 25, wherein the therapeutic agent is useful for treating diseases and disorders selected from the group consisting of addictive disorders, psychiatric disorders, infectious diseases, cardiovascular diseases, respiratory diseases, inflammatory conditions, immune-based diseases, auto-immune diseases, bone diseases and cancers.

32. The method according to claim 31, wherein the disorder is selected from the group consisting of narcotic addiction, alcohol addiction and craving and nicotine addition.

33. The method according to claim 32, wherein the disorder is narcotic addiction.

34. The method according to claim 33, wherein the therapeutic agent is a narcotic antagonist.

35. The method according to claim 34, wherein the narcotic antagonist is selected from the group consisting of naltrexone, naloxone, cyclazocine and nalmefene.

36. The method according to claim 35, wherein the narcotic antagonist is naltrexone.

37. A method of treating a patient in need of a therapeutic regimen, comprising the following steps:
administering at least one initial loading dosage form containing the therapeutic agent;
administering at least one receptor loading dosage form containing the therapeutic agent; and
administering at least one extended-release dosage form containing the therapeutic agent, wherein said initial loading dosage form, receptor loading dosage form and extended-release dosage form are administered substantially concurrently.

38. The method according to claim 37, wherein the at least one initial loading dosage form and the at least one receptor loading dosage form are independently selected from the group consisting of oral dosage forms, parenteral dosage forms (intramuscular or subcutaneous), intravenous dosage forms, a coating on the at least one extended-release dosage form and implantable dosage forms.

39. The method according to claim 38, wherein the at least one extended-release form is a non-erodible, implanted, subcutaneous dosage forms.

40. The method according to claim 39, wherein the non-erodible, implanted, subcutaneous dosage form is selected from the group consisting of hydrogels, silastic tubes and osmotic pumps.

41. The method according to claim 40, wherein the at least one extended release dosage form is a non-erodible, implanted, subcutaneous, hydrogel dosage form.

42. The method according to claim 41, wherein the at least one extended release dosage form is a non-erodible, implanted, subcutaneous, hydrogel dosage form fashioned from a co-polymer of various esters of methacrylic acid.

43. The method according to claim 37, wherein the therapeutic agent is useful for treating diseases and disorders selected from the group consisting of addictive disorders, psychiatric disorders, infectious diseases, cardiovascular diseases, respiratory diseases, inflammatory conditions, immune-based diseases, auto-immune diseases, bone diseases and cancers.

44. The method according to claim 43, wherein the disorder is selected from the group consisting of narcotic addiction, alcohol addiction and craving and nicotine addition.

45. The method according to claim 44, wherein the disorder is narcotic addiction.

46. The method according to claim 45, wherein the therapeutic agent is a narcotic antagonist.

47. The method according to claim 46, wherein the narcotic antagonist is selected from the group consisting of naltrexone, naloxone, cyclazocine and nalmefene.

48. The method according to claim 47, wherein the narcotic antagonist is naltrexone.

49. A method of delivering a therapeutic agent to a patient in need of the therapeutic agent, comprising the following steps:
determining an optimal dose and an optimal rate of delivery of the therapeutic agent sufficient to produce a minimum effective concentration of the therapeutic agent in the patient's bloodstream for a period of at least six months;
administering at least one receptor loading dosage form containing the therapeutic agent, wherein the at least one receptor loading dosage form releases the therapeutic agent at such a rate as to effectively load the patient's drug receptors until a steady state minimum effective concentration is achieved from at least one extended-release dosage form containing the therapeutic agent; and
administering the at least one extended-released dosage form containing the therapeutic agent, wherein the at least one receptor loading dosage form and the at least one extended-release dosage form are administered substantially concurrently.

50. The method according to claim 49, wherein the optimal rate of administration is calculated to be approximately equal to the rate at which the therapeutic agent leaves the receptors in the patient's body, at a steady state.

51. The method according to claim 50, wherein the at least one extended-release dosage form is adapted and configured to release the therapeutic agent in a substantially zero order manner.

52. The method according to claim 51, wherein the at least one extended-release dosage form is adapted and configured to release the therapeutic agent for about six to about twelve months.

53. The method according to claim 52, wherein the at least one receptor loading dosage form is selected from the group consisting of oral dosage forms, parenteral dosage forms (intramuscular or subcutaneous), intravenous dosage forms, a coating on the at least one extended-release dosage form and implantable dosage forms.

54. The method according to claim 53, wherein the at least one extended-release form is a non-erodible, implanted, subcutaneous dosage forms.

55. The method according to claim 54, wherein the non-erodible, implanted, subcutaneous dosage form is selected from the group consisting of hydrogels, silastic tubes and osmotic pumps.

56. The method according to claim 55, wherein the at least one extended release dosage form is a non-erodible, implanted, subcutaneous, hydrogel dosage form.

57. The method according to claim 56, wherein the at least one extended release dosage form is a non-erodible, implanted, subcutaneous, hydrogel dosage form fashioned from a co-polymer of various esters of methacrylic acid.

58. The method according to claim 49, wherein the therapeutic agent is useful for treating diseases and disorders selected from the group consisting of addictive disorders, psychiatric disorders, infectious diseases, cardiovascular diseases, respiratory diseases, inflammatory conditions, immune-based diseases, auto-immune diseases, bone diseases and cancers.

59. The method according to claim 58, wherein the disorder is selected from the group consisting of narcotic addiction, alcohol addiction and craving and nicotine addition.

60. The method according to claim 59, wherein the disorder is narcotic addiction.

61. The method according to claim 60, wherein the therapeutic agent is a narcotic antagonist.

62. The method according to claim 61, wherein the narcotic antagonist is selected from the group consisting of naltrexone, naloxone, cyclazocine and nalmefene.

63. The method according to claim 62, wherein the narcotic antagonist is naltrexone.

64. A method of delivering a therapeutic agent to a patient in need of the therapeutic agent, comprising the following steps:
determining an optimal dose and an optimal rate of delivery of the therapeutic agent sufficient to produce a minimum effective concentration of the therapeutic agent in the patient's bloodstream for a period of at least six months;
administering at least one initial loading dosage form containing the therapeutic agent;
administering at least one receptor loading dosage form containing the therapeutic agent, wherein the at least one receptor loading dosage form releases the therapeutic agent at such a rate as to effectively load the patient's drug receptors until a steady state minimum effective concentration is achieved from at least one extended-release dosage form containing the therapeutic agent; and
administering the at least one extended-released dosage form containing the therapeutic agent, wherein the at least one initial loading dosage form, the at least one receptor loading dosage form and the at least one extended-release dosage form are administered substantially concurrently.

65. The method according to claim 64, wherein the optimal rate of administration is calculated to be approximately equal to the rate at which the therapeutic agent leaves the receptors in the patient's body, at a steady state.

66. The method according to claim 65, wherein the at least one extended-release dosage form is adapted and configured to release the therapeutic agent in a substantially zero order manner.

67. The method according to claim 66, wherein the at least one extended-release dosage form is adapted and configured to release the therapeutic agent for about six to about twelve months.

68. The method according to claim 67, wherein the at least one initial loading dosage form and the at least one receptor loading dosage form are independently selected from the group consisting of oral dosage forms, parenteral dosage forms (intramuscular or subcutaneous), intravenous dosage forms, a coating on the at least one extended-release dosage form and implantable dosage forms.

69. The method according to claim 68, wherein the at least one extended-release form is a non-erodible, implanted, subcutaneous dosage forms.

70. The method according to claim 69, wherein the non-erodible, implanted, subcutaneous dosage form is selected from the group consisting of hydrogels, silastic tubes and osmotic pumps.

71. The method according to claim 70, wherein the at least one extended release dosage form is a non-erodible, implanted, subcutaneous, hydrogel dosage form.

72. The method according to claim 71, wherein the at least one extended release dosage form is a non-erodible, implanted, subcutaneous, hydrogel dosage form fashioned from a co-polymer of various esters of methacrylic acid.

73. The method according to claim 64, wherein the therapeutic agent is useful for treating diseases and disorders selected from the group consisting of addictive disorders, psychiatric disorders, infectious diseases, cardiovascular diseases, respiratory diseases, inflammatory conditions, immune-based diseases, auto-immune diseases, bone diseases and cancers.

74. The method according to claim 73, wherein the disorder is selected from the group consisting of narcotic addiction, alcohol addiction and craving and nicotine addition.

75. The method according to claim 74, wherein the disorder is narcotic addiction.

76. The method according to claim 75, wherein the therapeutic agent is a narcotic antagonist.

77. The method according to claim 76, wherein the narcotic antagonist is selected from the group consisting of naltrexone, naloxone, cyclazocine and nalmefene.

78. The method according to claim 77, wherein the narcotic antagonist is naltrexone.
